(19) United States
(12) Patent Application Publication
(10) Pub. No.: US 2011/0295047 A1
(43) Pub. Date: Dec. 1, 2011

(54) METHOD FOR PRODUCING COMPOUND

(75) Inventors: Masumi Itabashi, Kodaira-shi (JP); Hironobu Iwawaki, Yokohama-shi (JP)

(73) Assignee: CANON KABUSHIKI KAISHA, Tokyo (JP)

(21) Appl. No.: 13/131,109

(22) PCT Filed: Nov. 25, 2009
(86) PCT No.: PCT/JP2009/070204

§ 371 Date: Aug. 22, 2011

(30) Foreign Application Priority Data
Nov. 27, 2008 (JP) 2008-302433

(51) Int. Cl. C07C 2/00

(52) U.S. Cl. 585/320

(57) ABSTRACT

A method for producing a compound according to the present invention includes synthesizing a compound represented by general formula (3): R—Ar₁—X₁, which is an intermediate a, by subjecting a compound represented by general formula (1): Ar₁—X₁ and an alkyl bromide represented by general formula (2): R—Br to an alkylation reaction, using aluminum bromide as a catalyst; synthesizing a compound represented by general formula (4): R—Ar₁—X₂, which is an intermediate b, from the compound represented by general formula (3); and synthesizing a compound represented by general formula (5): R—Ar₁—Ar₂ from the compound represented by general formula (4).

### TECHNICAL FIELD

The present invention relates to a method for producing a compound.

### BACKGROUND ART

As a method of introducing an alkyl group into an aromatic compound, a Friedel-Crafts reaction (Non Patent Citation 1) is known and considered to be excellent in terms of high yields. The Friedel-Crafts reaction typically uses a chloride, such as aluminum chloride or iron chloride, which is a solid, as a catalyst, and an alkyl chloride as an alkylating agent.

Furthermore, typically, the reaction is solvent-free in which no solvent is used, or in view of the advantage that the catalyst is soluble, carbon disulfide, dichloromethane, or the like is used as a solvent (Patent Citation 1 and Non Patent Citation 2). In particular, Patent Citation 2 mentions an alkylation reaction using a Friedel-Crafts reaction in the process of synthesizing a fluorescent light-emitting material for an organic light-emitting device, the fluorescent light-emitting material having a skeleton composed of a fused polycyclic aromatic compound, such as perylene, decacyclene, or fluoranthene.

In Patent Citation 2, the alkylation reaction is carried out using, for example, aluminum chloride as a catalyst, although not particularly limited thereto, and an excess amount of tert-butyl chloride as an alkylating agent.

However, in the device using an organic light-emitting material produced by the production method including an alkylation reaction step using the reagents described above, the amount of attenuation of luminance is not always small and further improvement is desired.

### Patent Citation 1

Japanese Patent Laid-Open No. 2005-325097

### Patent Citation 2

Japanese Patent Laid-Open No. 9-241629

### Non Patent Citation 1

Shinjikken Kagaku Koza (New Experimental Chemical Course) 14-I, 62 (1977) Maruzen

### Non Patent Citation 2

L. A. Carpino et al. J. Org. Chem., 1989, 54, 4302

### DISCLOSURE OF INVENTION

The present invention provides a method for producing an organic compound having a pyrene ring having a tert-butyl group and not having chlorine during alkylation.

A method for producing a compound according to the present invention includes synthesizing a compound represented by general formula (3): R—Ar₁—X₁, which is an intermediate a, by subjecting a compound represented by general formula (1): Ar₁—X₁ and an alkyl bromide represented by general formula (2): R—Br to an alkylation reaction, using aluminum bromide as a catalyst; synthesizing a compound represented by general formula (4): R—Ar₁—X₂, which is an intermediate b, from the compound represented by general formula (3); and synthesizing a compound represented by general formula (5): R—Ar₁—Ar₂ from the compound represented by general formula (4), wherein Ar₁ is a substituted or unsubstituted pyrene ring; X₁ is a hydrogen atom or a halogen atom; Ar₂ is a substituted or unsubstituted phenyl group, or a substituted or unsubstituted fused polycyclic aromatic group; X₂ is a boronic acid group or a boronic ester group; and R is a tert-butyl group.

According to the present invention, it is possible to produce an organic compound having a pyrene ring having a tert-butyl group and not having chlorine during alkylation. Consequently, by using the thus obtained compound for an organic light-emitting device, it is possible to provide a device in which the amount of attenuation of luminance is small when an operation is performed for a long period of time.

### DESCRIPTION OF EMBODIMENTS

The compound obtained by the production method of the present invention is represented by general formula (5): R—Ar₁—Ar₂, wherein Ar₁ is a substituted or unsubstituted pyrene ring; Ar₂ is a substituted or unsubstituted phenyl group, or a substituted or unsubstituted fused polycyclic aromatic group; and R is a tert-butyl group.

Specifically, R is a tert-butyl group, Ar₁ is a pyrene ring, and Ar₂ is a naphthalene ring.

When the compound represented by general formula (5) is synthesized, a compound represented by general formula (4): R—Ar₁—X₂ is synthesized in advance as a starting material for the compound represented by general formula (5). The compound represented by general formula (4) is defined as an intermediate b. In general formula (4), X₂ is a boronic acid group or a boronic ester group. The intermediate b is synthesized from an intermediate a. The intermediate a is represented by general formula (3): R—Ar₁—X₁, wherein X₁ is a hydrogen atom or a halogen atom. Furthermore, the intermediate a is obtained by alkylating a compound represented by general formula (1): Ar₁—X₁. That is, the reaction path for synthesizing the compound represented by general formula (5) from the compound represented by general formula (1) is shown as below.

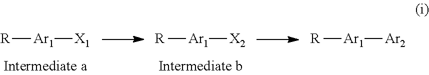

In the method for producing the compound according to the present invention, in the alkylation reaction in which the intermediate a represented by general formula (3) is obtained from the compound represented by general formula (1), all of the following conditions must be satisfied: 1) as a compound that reacts with the compound represented by general formula (1), a bromine-containing compound can be used, but a chlorine-containing compound cannot be used; 2) as a catalyst, a bromine-containing compound can be used, but a chlorine-containing compound cannot be used; and 3) a solvent is not used, or when a solvent is used, a non-halogen solvent is used.

That is, in the alkylation reaction, among halogens, bromine may be present, but it is important that chlorine is not present in the reaction environment.

Accordingly, in the method for producing the compound according to the present invention, in the synthesis of the intermediate a, which is a first step in synthesizing the compound represented by general formula (5), i.e., in the alkylation reaction, the following three conditions, which show the schemes described above more specifically, are required: 1) as a compound allowed to react with the compound represented by general formula (1), an alkyl bromide is used; 2) as a catalyst, aluminum bromide is used; and 3) a solvent is not used, or when a solvent is used, a non-halogen solvent is used. In the alkylation reaction carried out under the conditions described above, a by-product which is analogous to the intermediate a to which chlorine is added (hereinafter referred to as a “chlorine adduct”) is not generated.

The chlorine adduct which is analogous to the intermediate a has a similar structure as that of the intermediate a. Therefore, after the reaction, it is very difficult to separate the two from each other by usual purification procedures. In the present invention, attention has been focused on completely removing room for generation of such a chlorine adduct which is analogous to the intermediate a.

Using an organic light-emitting device having a compound represented by general formula (5) synthesized from an intermediate a free from a chlorine adduct which is analogous to the intermediate a, it is possible to provide an organic light-emitting device in which the amount of attenuation of luminance is small even when an operation is performed for a long period of time. Furthermore, it has been found that, when an alkyl bromide and aluminum bromide are used, the compound represented by general formula (5) does not even contain bromine, which is an unexpected effect.

Furthermore, the intermediate a synthesized by the production method according to the present invention is purified by a known purification process after the alkylation reaction. The known purification process is, for example, silica gel column chromatography or recrystallization.

The fact that the resulting intermediate a does not contain chlorine can be confirmed by measuring the concentration of chlorine contained in the intermediate a. The fact that the resulting intermediate a does not contain chlorine means that the chlorine concentration is in a range from 1.0 ppm or less to the detection limit of the detector. In addition, as long as chlorine is not present in the alkylation reaction, a chlorine-containing compound may be used in a reaction subsequent to the alkylation reaction.

The non-halogen solvent described in this description is a solvent composed of an organic compound which does not contain a halogen atom as a component, and any of aliphatic solvents and aromatic solvents may be used. Use of an aliphatic solvent is more desirable from the standpoint that a side reaction of alkylation of the solvent does not occur. Furthermore, whether a solvent is used or not used, any halogen as an impurity must be prevented from being mixed. In this description, the halogen is any of chlorine, bromine, and iodine.

The alkylation reaction will be further described below.

An example of a reaction formula in which a compound represented by general formula (3), i.e., an intermediate a, is obtained from a compound represented by general formula (1) is shown below.

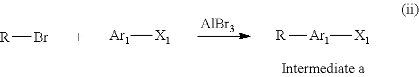

As shown above, the intermediate a is obtained by alkylation from general formula (1). Two reaction formulae are shown below as specific examples.

### Specific Example 1

### X₁=H

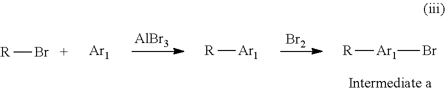

### Specific Example 2

### X₁=Br

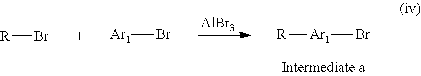

So far, i.e., up to the alkylation reaction, chlorine is not used.

Next, the path for synthesizing an intermediate b from the intermediate a is described. A specific example of the synthesis path is shown below. Here, as shown in the reaction formula, a chlorine-containing compound may be used.

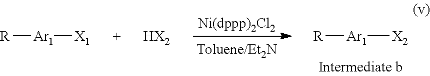

Next, the path for synthesizing a compound represented by general formula (5), which is an end product, from the intermediate b is described. A specific example of the synthesis path is shown below.

The compound represented by general formula (5), which is an end product, can be obtained by coupling the intermediate b with an intermediate c.

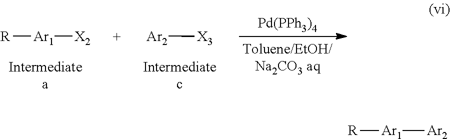

In the reaction formula (vi), X₃ is a halogen atom or a triflate group.

For the purpose of reference, the following will be described although it is not a production method according to the present invention.

The coupling reaction shown in the reaction formula (vi) can also be carried out using an intermediate d represented by general formula (6): R—Ar₁—X₃ and an intermediate e represented by general formula (7): Ar₂—X₂, in which the reactive moieties X₂ and X₃ are added the other way around.

Furthermore, for the purpose of reference, the following will be described although it is not a production method according to the present invention.

That is, by using the alkylation reaction described above, various compounds can be produced. When such compounds are expressed, for example, using general formula (5) of the present invention, examples of R, Ar₁, and Ar₂ can be mentioned as below.

Examples of R include an iso-propyl group.

Examples of Ar₁ include a fluorene ring, a perylene ring, a fluoranthene ring, a chrysene ring, and an anthracene ring.

Examples of Ar₂ include a benzene ring, a phenanthrene ring, a fluoranthene ring, a pyrene ring, a chrysene ring, a perylene ring, a fluorene ring, and an anthracene ring.

In this case, Ar₂ may have another substituent thereon. For example, when Ar₂ is a naphthalene ring, a fluorene ring may bind to the naphthalene ring. In such a case, the naphthalene ring binds to Ar₁.

Referring back to the present invention, description will be made below.

An organic light-emitting device includes at least an anode, a cathode, and an organic compound layer disposed between the anode and the cathode. In the organic light-emitting device, when electric charge is supplied between the anode and the cathode, an organic compound constituting the organic compound layer or an organic compound contained in the organic compound layer emits light. Since the organic light-emitting device emits light, the organic compound layer corresponds to a light-emitting layer or a light-emitting region.

The organic light-emitting device may include another layer other than the organic compound layer. The other layer may be an inorganic compound layer or an organic compound layer.

Examples of the other layer include a hole injection layer, a hole transport layer, an electron blocking layer, a hole blocking layer, an electron transport layer, and an electron injection layer. These layers may be appropriately disposed between the anode and the cathode.

Each of the anode and the cathode may be appropriately composed of a suitable material. The electrode which is disposed on the side from which light is extracted out of the organic light-emitting device is semi-transmissive or transmissive to the light. For example, ITO can be used.

In the case where it is required to reflect light in the organic light-emitting device, a highly reflective material is suitably used. Examples of such a material include silver and aluminum.

Furthermore, even in the case where it is required to reflect light in the organic light-emitting device, a structure may be employed in which an electrode composed of a semi-transmissive or transmissive material is provided on the reflecting side, and a reflecting member is separately arranged.

The organic light-emitting device may be driven by an active matrix driving method or a passive matrix driving method. In the case of the active matrix driving method, a driving circuit that drives the organic light-emitting device includes a TFT, a capacitor, etc.

A plurality of organic light-emitting devices as luminous points can be integrated and used, for example, as a lighting apparatus. Furthermore, using luminous points as pixels, organic light-emitting devices can be used for a display part of a display device. Such a display device can be used for a PC display, a television, or an image pickup apparatus.

Examples of the image pickup apparatus include a digital video camera and a digital still camera. The image pickup apparatus includes an image display part referred to as a “finder”. A display part having organic light-emitting devices can be used for the image display part.

Furthermore, a display part having organic light-emitting devices can be used for an operation panel of any of various electrical apparatuses, etc.

Furthermore, in an electrophotography-type image forming apparatus, such as a laser printer or a copying machine, organic light-emitting devices can be used as a light source for exposing a photosensitive member. The light source can have a structure in which a plurality of organic light-emitting devices are arranged in the longitudinal direction of the photosensitive member.

As described above, organic light-emitting devices can be suitably used for various apparatuses. For that purpose, it is also necessary to provide organic light-emitting devices in which the amount of attenuation of luminance is small even when an operation is performed for a long period of time, and compounds obtained by the method for producing a compound according to the present invention can be suitably used.

### Examples

Examples will be described below.

Synthesis Examples 1 to 10 are examples in which the alkylation reaction step is carried out in the absence of a chlorine-containing compound. In contrast, Comparative Synthesis Examples 1 to 13 are examples in which the reaction step is carried out in the presence of a chlorine-containing compound.

As is evident from the examples, by eliminating a chlorine-containing compound from the step of carrying out an alkylation reaction, it is possible to prevent generation of a chlorine adduct, which is a by-product, in the synthesis of the intermediate a regardless of the amounts of an alkylating agent and a catalyst.

Moreover, as is evident from Synthesis Examples 1 to 10, even if a solvent is not used, the intermediate a can be obtained in high yields. Furthermore, Device Examples 1 to 10 show the relative luminance ratio of organic light-emitting devices containing compounds represented by general formula (5) obtained from the intermediates a of Synthesis Examples 1 to 10. Device Comparative Examples 1 to 13 show the relative luminance ratio of organic light-emitting devices containing compounds obtained from Comparative Synthesis Examples 1 to 13.

In each of Synthesis Examples and Comparative Examples, bromine is hardly detected. Meanwhile, the compounds used in Device Comparative Examples 1 to 13 contain chlorine, and the relative luminance ratio of organic light-emitting devices is low. In contrast, the organic light-emitting devices containing the compounds described in Device Examples 1 to 10 each have a high relative luminance ratio, and it is possible to provide organic light-emitting devices in which the amount of attenuation of luminance is small even when an operation is performed for a long period of time.

### Synthesis Examples of Intermediate a by Alkylation Reaction

### Synthesis Example 1

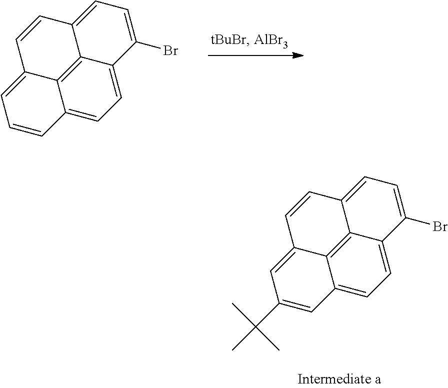

In a reaction container, 3.95 g (0.0141 mmol) of 1-bromopyrene and 138 ml (1.23 mol, 87.8 equivalents) of tert-butyl bromide were placed, under stirring at 0° C., aluminum bromide was added thereto, and stirring was performed at 0° C. for 30 minutes (conversion rate 93.7%). Next, 5.46 ml (3.2 eq vs. aluminum bromide) of ethanol was added, and 31.2 ml (8 eq vs. aluminum bromide) of triethylamine was further added thereto, followed by stirring for 10 minutes. Then, the resulting organic layer was washed with pure water and isolated. The organic layer was dried over sodium sulfate, and then concentrated. The resulting solid was purified by silica gel column chromatography and concentrated. The resulting powder was dispersed and washed in a mixed solution of ethanol/heptane, and left to cool, followed by filtration to give 2.97 g of 1-bromo-7-tert-butylpyrene as an intermediate a (yield 62.7%, purity 98.7%).

### Synthesis Example 2

1-Bromo-7-tert-butylpyrene was obtained as in Synthesis Example 1 except that the equivalent ratio of tert-butyl bromide to 1-bromopyrene was changed as shown in Table 1. The results are shown in Table 1.

### Synthesis Examples 3 to 10 and Comparative Synthesis Examples 1 to 13

1-Bromo-7-tert-butylpyrene was obtained as in Synthesis Example 1 except that the equivalent ratios of tert-butyl bromide and a solvent (hexane, dichloromethane, toluene, or 1,2-dichlorobenzene) to 1-bromopyrene were changed as shown in Table 1. In the example in which the equivalent ratio of the solvent is not described, the amount of the solvent used is twenty times the mass of 1-bromopyrene. The results are shown in Table 1.

TABLE 1

| | Alkylating agent | | Catalyst | | | | | | Concentration of |
|---|---|---|---|---|---|---|---|---|---|
| | (equivalent) | | (equivalent) | | | | Conversion | Yield | Cl contained |
| | tBuCl | tBuBr | AlCl₃ | AlBr₃ | Solvent (equivalent) | | rate (%) | (%) | (ppm) |
| Synthesis | — | 87.9 | — | 2.0 | — | | 93.7 | 62.7 | N.D. |
| Example 1 | | | | | | | | | |
| Synthesis | — | 50.0 | — | 2.0 | — | | 89.9 | 63.1 | 0.3 |
| Example 2 | | | | | | | | | |
| Synthesis | — | 87.8 | — | 2.0 | n-Hexane | 1.0 | 93.6 | 61.6 | N.D. |
| Example 3 | | | | | | | | | |
| Synthesis | — | 86.7 | — | 2.0 | n-Hexane | 1.0 | 92.8 | 59.9 | N.D. |
| Example 4 | | | | | | | | | |
| Synthesis | — | 70.0 | — | 2.0 | n-Hexane | 15.0 | 87.2 | 62.5 | N.D. |
| Example 5 | | | | | | | | | |
| Synthesis | — | 65.7 | — | 2.0 | n-Hexane | 18.8 | 85.6 | 59.1 | N.D. |
| Example 6 | | | | | | | | | |
| Synthesis | — | 87.8 | — | 2.0 | n-Hexane | 37.7 | 83.3 | 57.8 | 0.2 |
| Example 7 | | | | | | | | | |
| Synthesis | — | 43.9 | — | 2.0 | n-Hexane | 37.7 | 44.6 | 28.7 | N.D. |
| Example 8 | | | | | | | | | |
| Synthesis | — | 4.0 | — | 2.0 | n-Hexane | 79.8 | 36.0 | 23.9 | N.D. |
| Example 9 | | | | | | | | | |
| Synthesis | — | 87.7 | — | 2.0 | Toluene | 1.0 | 61.2 | 40.8 | 0.6 |
| Example 10 | | | | | | | | | |
| C. Synthesis | 2.0 | — | 1.0 | — | Dichloromethane | | 97.3 | 75.2 | 234.9 |
| Example 1 | | | | | | | | | |
| C. Synthesis | 87.8 | — | 2.0 | — | — | | 98.1 | 74.3 | 21.2 |
| Example 2 | | | | | | | | | |
| C. Synthesis | 87.7 | — | — | 2.0 | — | | 96.5 | 72.1 | 8.1 |
| Example 3 | | | | | | | | | |
| C. Synthesis | — | 87.8 | 2.0 | — | — | | 95.1 | 73.6 | 9.5 |
| Example 4 | | | | | | | | | |
| C. Synthesis | 2.0 | — | — | 1.0 | Dichloromethane | | 94.1 | 71.5 | 6.9 |
| Example 5 | | | | | | | | | |
| C. Synthesis | — | 4.0 | 2.0 | — | Dichloromethane | | 93.2 | 76.8 | 13.3 |
| Example 6 | | | | | | | | | |
| C. Synthesis | — | 1.2 | — | 1.1 | Dichloromethane | | 85.4 | 61.3 | 20.1 |
| Example 7 | | | | | | | | | |
| C. Synthesis | 4.0 | — | — | 1.0 | 1,2-Dichlorobenzene | | 94.1 | 63.4 | 9.0 |
| Example 8 | | | | | | | | | |
| C. Synthesis | — | 4.0 | — | 1.0 | 1,2-Dichlorobenzene | | 64.9 | 52.7 | 3.4 |
| Example 9 | | | | | | | | | |
| C. Synthesis | — | 87.7 | — | 2.0 | Dichloro- | 1.0 | 92.1 | 62.5 | 1.9 |
| Example 10 | | | | | methane | | | | |
| C. Synthesis | 87.7 | — | 2.0 | — | Hexane | 1.0 | 97.9 | 72.1 | 11.2 |
| Example 11 | | | | | | | | | |
| C. Synthesis | 87.8 | — | — | 2.0 | Hexane | 1.0 | 95.0 | 69.8 | 8.3 |
| Example 12 | | | | | | | | | |
| C. Synthesis | — | 87.7 | 2.0 | — | Hexane | 1.0 | 93.8 | 70.1 | 10.4 |
| Example 13 | | | | | | | | | |

In Table 1, “tBu” represents a tert-butyl group. The amount of each reagent is the equivalent ratio to 1-bromopyrene. “C. Synthesis Example” means Comparative Synthesis Example.

Next, the concentration of chlorine contained in 1-bromo-7-tert-butylpyrene obtained by the alkylation reaction step of the present invention was analyzed by combustion ion chromatography.

The analysis was performed using an apparatus obtained by combining an automatic sample combustion device AQF-100 manufactured by Dia Instruments Co., Ltd. and an ion chromatography system ICS-1500 manufactured by Dionex Corporation.

First, a bromine ion calibration curve was prepared using sodium bromide as internal standard ions, and a chlorine ion calibration curve was prepared using sodium chloride. Next, 30 mg of an actual sample was completely burned using the combustion device, and absorbed by an absorbing solution prepared by diluting hydrogen peroxide with ultrapure water (concentration 30 ppm). The resulting solution was analyzed and measured by the ion chromatography system. Finally, by subtracting the chlorine ion concentration and the bromine ion concentration in a blank sample from the concentrations measured from the actual sample, the concentration of halogen ions contained in the sample was calculated.

The concentration of chlorine contained in the intermediate a (1-bromo-7-tert-butylpyrene) obtained by the alkylation reaction step according to the present invention was 1 ppm or less. Table 1 shows a part thereof.

In contrast, in each of the compounds obtained in Comparative Synthesis Examples, the concentration of chlorine contained exceeded 1 ppm. In addition, although Comparative Synthesis Examples 1 to 13 each showed a high conversion rate, the detected concentration of chlorine contained was several ppm to several hundred ppm.

Furthermore, it has been confirmed that, when a solvent is not used in the alkylation reaction, by using tert-butyl bromide in an amount of 50 equivalents or more to the compound represented by general formula (1), more specifically, 1-bromopyrene, the intermediate a can be obtained in high conversion rate, thus being desirable.

### Synthesis Example of Intermediate b

### Synthesis Example 11

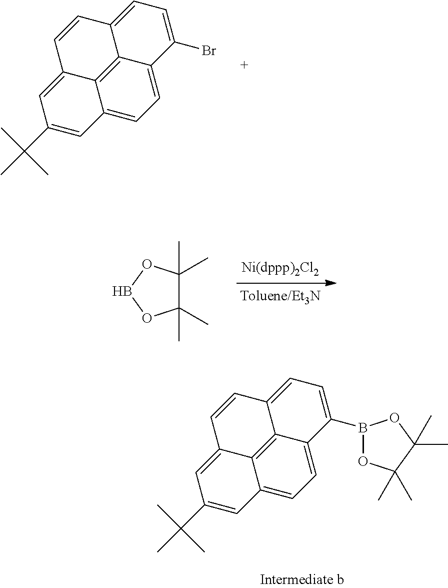

In a reaction container, under a nitrogen stream, 1.00 g (2.97 mmol) of the intermediate a (1-bromo-7-tert-butlpyrene) synthesized in Synthesis Example 1, 0.321 g (0.590 mmol, 0.2 eq) of [1,3-bis(diphenylphosphino)propane]-dichloronickel, 30 ml of toluene (dehydrated), 1.23 ml (8.90 mmol, 3 eq) of triethylamine, and 1.29 ml (8.90 mmol, 3 eq) of 4,4,5,5-tetramethyl-[1,3,2]dioxaborane were placed, and stirring was performed under heating at 90° C. for 6 hours. Next, pure water was added thereto, followed by stirring. Then, solids were removed by filtration, and an organic layer was isolated. The organic layer was dried over sodium sulfate, followed by concentration to give crude crystals. The crude crystals were purified by silica gel column chromatography, concentrated, and then dispersed and washed in a mixed solution of ethanol/methanol, followed by filtration to give 0.63 g of an intermediate b (yield 54.9%).

Furthermore, using each of the intermediates a synthesized in Synthesis Examples 2 to 10, an intermediate b was synthesized.

Furthermore, using each of the chlorine-containing intermediates a synthesized in Comparative Synthesis Examples 1 to 13, an intermediate b was synthesized.

### Synthesis Example of Intermediate c

### Synthesis Example 12

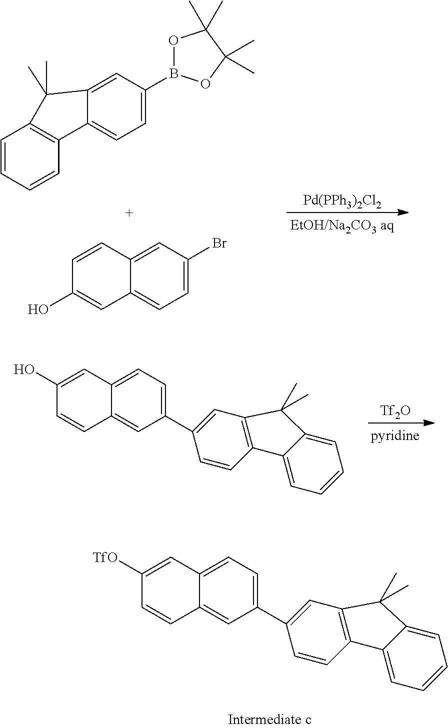

In a reaction container, 3.17 g (14.2 mmol) of 6-bromo-2-naphthol, 5.00 g (15.6 mmol) of 2-(9,9-dimethyl-9H-fluoren-2-yl)-4,4,5,5-tetramethyl-[1,3,2]doxaborane, 96.0 ml of ethanol, 6.78 g (21.3 mmol) of sodium carbonate, 48.0 ml of pure water, and 30 mg (14.2×10⁻³ mmol) of Pd(PPh₃)₂Cl₂ were placed, and heat-refluxing was performed for 4 hours. After cooling, pure water was added thereto, and filtration was performed to give crude crystals. The resulting crude crystals were washed with pure water and heptane, and thereby 3.94 g (yield 82.5%) of fluorenyl naphthol was obtained. Next, 10.5 g (31.2 mmol) of fluorenyl naphthol and 100 ml of pyridine were placed in a reaction container, and 15.5 ml (93.6 mmol) of trifluoromethanesulfonic anhydride was added dropwise thereto in an ice bath. Then, stirring was performed for 3 hours, and the reaction solution was poured into ice water, followed by filtration. The resulting crude crystals were washed with methanol to thereby obtain 6.99 g (yield 64.7%) of an intermediate c.

### Synthesis Example 13

### Synthesis Example of Compound A as Example of End Product Represented by General Formula (5)

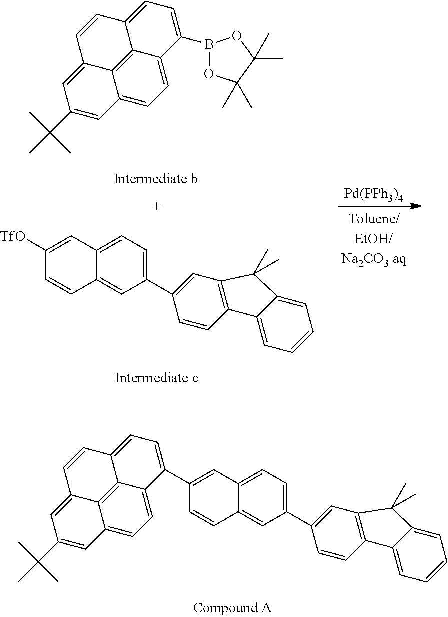

In a reaction container, 7.63 g (19.8 mmol) of the intermediate b obtained in Synthesis Example 11, 8.45 g (18.0 mmol) of the intermediate c, 0.63 g (0.54 mmol) of Pd(PPh₃)₄, 3.82 g (36.1 mmol) of sodium carbonate, 126.8 ml of toluene, 25.4 ml of ethanol, and 25.4 ml of pure water were placed, and heat-refluxing was performed for one hour. After cooling, ethanol was added thereto, and filtration was performed to give crude crystals. The resulting crude crystals were washed with pure water, followed by purification by silica gel column chromatography to give 6.16 g (yield 72.8%) of the intended compound A. The resulting compound A was purified by sublimation.

The structure was confirmed by NMR measurement. The attribution of peaks is as follows:

¹H-NMR (500 MHz, CDCl₃): δ (ppm)=8.25-8.21 (m, 5H), 8.12-8.10 (m, 4H), 8.07-8.01 (m, 3H), 7.91 (dd, 1H), 7.87 (D, 1H), 7.84-7.76 (m, 4H), 7.48 (D, 1H), 7.40-7.33 (m, 2H), 1.60 (S, 6H), 1.59 (S, 9H)

Furthermore, using each of the intermediates b synthesized from the intermediates a synthesized in Synthesis Examples 2 to 10, a compound A was synthesized.

Furthermore, using each of the intermediates b synthesized from the chlorine-containing intermediates a synthesized in Comparative Synthesis Examples 1 to 13, a compound A was synthesized.

### Example

An example of a production method according to the present invention is constituted of a synthesis example of the intermediate a by the alkylation reaction described above, a synthesis example of the intermediate b, and a synthesis example of the compound A, which is an example of the compound represented by general formula (5), i.e., the end product.

### Device Production Examples

### Device Examples 1 to 10 and Device Comparative Examples 1 to 13

An anode was formed by depositing indium tin oxide (ITO) by sputtering on a glass substrate. The thickness of the anode was set at 120 nm. Next, the substrate provided with the anode was subjected to ultrasonic cleaning using acetone and isopropyl alcohol (IPA) in that order, and then washed with pure water, followed by drying. Next, the substrate was subjected to UV/ozone cleaning, and the cleaned substrate was used as a transparent, conductive support substrate.

Next, as a hole injection material, the compound B-1 shown below and chloroform were mixed to prepare a 0.1% by weight chloroform solution.

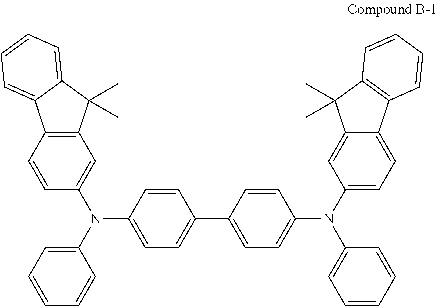

The chloroform solution was added dropwise onto the anode, and a film was formed by spin-coating, first at 500 rpm for 10 seconds, and then at 1,000 rpm for 40 seconds. Subsequently, drying was performed in a vacuum oven at 80° C. for 10 minutes to completely remove the solvents from the thin film. Thereby, a hole injection layer was formed. The thickness of the hole injection layer was 15 nm.

Next, the compound B-2 shown below was deposited by a vacuum vapor deposition method on the hole injection layer. Thereby, a hole transport layer was formed. The thickness of the hole transport layer was set at 15 nm.

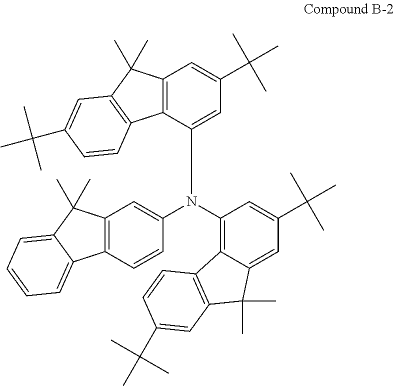

Next, the compound B-3 shown below as a guest (light-emitting material) and the compound A as a host were co-vapor-deposited by a vacuum vapor deposition method on the hole transport layer such that the weight ratio was 5:95. Thereby, a light-emitting layer was formed. In this step, the deposition was performed under the conditions where the thickness of the light-emitting layer was 30 nm, the degree of vacuum during vapor deposition was 1.0×10⁻⁴ Pa, and the deposition rate was 0.1 to 0.2 nm/sec.

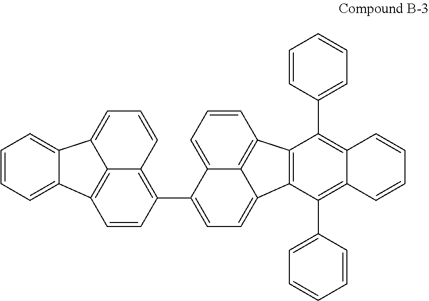

Next, 2,9-bis[2-(9,9′-dimethylfluorenyl)]-1,10-phenanthroline not containing a halogen substituent was deposited by a vacuum vapor deposition method on the light-emitting layer. Thereby, an electron transport layer was formed. In this step, the deposition was performed under the conditions where the thickness of the electron transport layer was 30 nm, the degree of vacuum during vapor deposition was 1.0×10⁻⁴ Pa, and the deposition rate was 0.1 to 0.2 nm/sec.

Next, lithium fluoride (LiF) was deposited by a vacuum vapor deposition method on the electron transport layer. Thereby, an electron injection layer was formed. In this step, the deposition was performed under the conditions where the thickness of the electron injection layer was 0.5 nm, the degree of vacuum during vapor deposition was 1.0×10⁻⁴ Pa, and the deposition rate was 0.01 nm/sec. Next, an aluminum film was formed by a vacuum vapor deposition method. Thereby, a cathode was formed. In this step, the deposition was performed under the conditions where the thickness of the cathode was 150 nm, the degree of vacuum during vapor deposition was 1.0×10⁻⁴ Pa, and the deposition rate was 0.5 to 1.0 nm/sec.

Next, a protective glass plate was placed over the workpiece in a dry air atmosphere and sealing was performed using an acrylic resin adhesive so that the device was prevented from being degraded due to adsorption of water. Thus, an organic light-emitting device was obtained.

Table 2 below shows the concentration of halogens contained during the alkylation step in the production process of the compound A which is an organic light-emitting material, and the influence on the device characteristics. The halogen concentration was measured by the combustion ion chromatography described above.

In Table 2, the device examples are numbered so as to correspond to the synthesis examples. That is, a compound A was synthesized for each of the intermediates a obtained from the synthesis examples. Organic light-emitting devices were fabricated using the respective compounds A. Device example numbers are assigned to the individual organic light-emitting devices.

TABLE 2

| | | | | Relative luminance |
|---|---|---|---|---|
| | Compound A | Halogen concentration (ppm) | | ratio |
| | Alkylation step | Cl | Br | L/L₀ |
| Device Example 1 | Synthesis Example 1 | N.D. | N.D. | 0.94 |
| Device Example 2 | Synthesis Example 2 | N.D. | N.D. | 0.94 |
| Device Example 3 | Synthesis Example 3 | N.D. | N.D. | 0.95 |
| Device Example 4 | Synthesis Example 4 | N.D. | N.D. | 0.91 |
| Device Example 5 | Synthesis Example 5 | N.D. | N.D. | 0.91 |
| Device Example 6 | Synthesis Example 6 | N.D. | N.D. | 0.92 |
| Device Example 7 | Synthesis Example 7 | 0.3 | N.D. | 0.91 |
| Device Example 8 | Synthesis Example 8 | N.D. | N.D. | 0.93 |
| Device Example 9 | Synthesis Example 9 | N.D. | N.D. | 0.92 |
| Device Example 10 | Synthesis Example 10 | 0.5 | 0.5 | 0.90 |
| Device Comparative | Comparative Synthesis | 30.0 | N.D. | 0.55 |
| Example 1 | Example 1 | | | |
| Device Comparative | Comparative Synthesis | 19.8 | N.D. | 0.61 |
| Example 2 | Example 2 | | | |
| Device Comparative | Comparative Synthesis | 5.1 | N.D. | 0.78 |
| Example 3 | Example 3 | | | |
| Device Comparative | Comparative Synthesis | 5.5 | N.D. | 0.76 |
| Example 4 | Example 4 | | | |
| Device Comparative | Comparative Synthesis | 6.8 | N.D. | 0.71 |
| Example 5 | Example 5 | | | |
| Device Comparative | Comparative Synthesis | 9.8 | N.D. | 0.73 |
| Example 6 | Example 6 | | | |
| Device Comparative | Comparative Synthesis | 20.0 | 0.8 | 0.60 |
| Example 7 | Example 7 | | | |
| Device Comparative | Comparative Synthesis | 8.6 | N.D. | 0.73 |
| Example 8 | Example 8 | | | |
| Device Comparative | Comparative Synthesis | 2.3 | 1.3 | 0.81 |
| Example 9 | Example 9 | | | |
| Device Comparative | Comparative Synthesis | 1.5 | N.D. | 0.83 |
| Example 10 | Example 10 | | | |
| Device Comparative | Comparative Synthesis | 10.2 | N.D. | 0.70 |
| Example 11 | Example 11 | | | |
| Device Comparative | Comparative Synthesis | 9.0 | N.D. | 0.73 |
| Example 12 | Example 12 | | | |
| Device Comparative | Comparative Synthesis | 9.9 | N.D. | 0.70 |
| Example 13 | Example 13 | | | |

Here, the ratio L/Lₒ is a numerical value showing the relative luminance ratio of the luminance after 100 hours to the initial luminance, in which the luminance is detected by a photodiode when the fabricated organic light-emitting device is continuously driven at constant current (100 mA/cm²). Consequently, as the numerical value is closer to 1.0, the degree of degradation is smaller, indicating an organic light-emitting device in which the amount of attenuation of luminance is small even when an operation is performed for a long period of time.

In the compound A synthesized by the alkylation step according to the present invention, the concentration of chlorine contained is 1 ppm or less. This shows that the organic light-emitting device using the compound A is less easily degraded than the organic light-emitting device using the compound synthesized by the conventional alkylation step. The results reveal a correlation between the concentration of chlorine contained in the compound A and the relative luminance ratio of the organic light-emitting device using the compound A. More specifically, as the concentration of chlorine contained decreases, the relative luminance ratio improves. That is, by producing an organic light-emitting material using an alkylation reaction step in which a halogen adduct is not generated as a by-product, it is possible to provide organic light-emitting devices in which the amount of attenuation of luminance is small even when an operation is performed for a long period of time.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-302433, filed Nov. 27, 2008, which is hereby incorporated by reference herein in its entirety.

1. A method for producing a compound comprising:
synthesizing a compound represented by general formula (3): R—Ar₁—X₁, which is an intermediate a, by subjecting a compound represented by general formula (1): Ar₁—X₁ and an alkyl bromide represented by general formula (2): R—Br to an alkylation reaction, using aluminum bromide as a catalyst;
synthesizing a compound represented by general formula (4): R—Ar₁—X₂, which is an intermediate b, from the compound represented by general formula (3); and
synthesizing a compound represented by general formula (5): R—Ar₁—Ar₂ from the compound represented by general formula (4),
wherein Ar₁ is a pyrene ring; X₁ is a bromide atom; Ar₂ is a substituted or unsubstituted phenyl group, or a substituted or unsubstituted fused polycyclic aromatic group; X₂ is a boronic acid group or a boronic ester group; and R is a tert-butyl group, and
wherein Ar₁—X₁ is represented by the following structural formula:

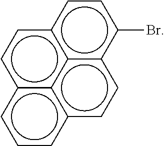

2. The method for producing a compound according to claim 1, wherein, in the alkylation reaction, the compound represented by general formula (1), the alkyl bromide, and the aluminum bromide only are used.

3. The method for producing a compound according to claim 1, wherein, in the alkylation reaction, in addition to the compound represented by general formula (1), the alkyl bromide, and the aluminum bromide, a non-halogen solvent is used.

4. The method for producing a compound according to claim 2, wherein, in the alkylation reaction, the alkyl bromide is used in an amount of 50 equivalents or more to the compound represented by general formula (1).
